(19) United States
(12) Patent Application Publication
(10) Pub. No.: US 2011/0003751 A1
(43) Pub. Date: Jan. 6, 2011

(54) Novel Immunoregulatory Peptides, Compositions and Uses Thereof

(76) Inventor: Vladislav I. Deigin, Richmond Hill (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/743,221

(22) PCT Filed: Nov. 20, 2008
(86) PCT No.: PCT/CA2008/002037

§ 371 Date: Sep. 17, 2010

Related U.S. Application Data
(60) Provisional application No. 60/989,280, filed on Nov. 20, 2007.

(51) Int. Cl. A61K 38/06; C07K 5/08; A61P 35/00; A61P 35/02; A61P 37/00

(52) U.S. Cl. 514/13.5; 530/331; 514/21.9; 514/19.3; 514/19.6

(57) ABSTRACT

A biologically active peptide, of Formula (1) [X-L-γ-Glu-Trp-Y], a novel pharmaceutical composition containing the peptide, a method for preparing the peptide and uses of the peptide are disclosed.

### FIELD

The disclosure relates to immunoregulatory peptides, a pharmaceutical composition containing the peptides and uses thereof.

### BACKGROUND

In practical medicine, thymic extracts, among them thymosin fraction 5 and thymalin (U.S. Pat. No. 5,070,026) are widely known regulators of immune processes. The extracts consist of a variety of polypeptides. Their production from natural sources is limited by the complicated production process, low yields of active substances and a considerable variability of their physicochemical parameters and biological properties. Besides, the ballast components present in natural thymic preparations may cause side effects in patients. This latter fact has spurred the development of synthetic peptides. A number of peptides with immunoregulatory effects have been previously synthesized: SU Patent No. 1582393, EP Patent No. 230052, U.S. Pat. No. 5,008,246 and U.S. Pat. No. 5,013,723. There are also patents (U.S. Pat. No. 6,051,683, U.S. Pat. No. 6,159,940 and WO 9640740) describing the peptide H-Ile-Glu-Trp-Y, where Y is OH or a substituted amide (C₁-C₃).

Each of the synthesized peptides has a certain set of necessary properties, is highly active, low toxic, has no side effects, and may therefore be used in medicine. One of the limitations for wide medical use of synthetic peptides is their instability when administered by oral route. There are different methods for improving enzymatic stability of a peptide. One of them was proposed by Kolobov et al. (U.S. Pat. No. 5,916,878 and U.S. Pat. No. 5,744,452).

### SUMMARY

The present application describes highly active peptides having immunoregulatory properties and improved oral activity. The identified peptides have the ability to effectively modulate the immune system of humans and animals, in relatively low doses, and with little or no side effects. It has been found that these peptides have hemopoietic properties.

Accordingly, the present disclosure includes a compound selected from a compound of the Formula I:

X-L-γ-Glu-Trp-Y (I)

wherein
X is selected from H, C(O)C₁₋₄alkyl, L-Leu, L-Ile and L-Trp;
Y is selected from OH, NH₂, NHC₁₋₄alkyl, N(C₁₋₄alkyl)(Cᵢ₋₄alkyl), L-Leu and L-Ile; and
and pharmaceutically acceptable salts, solvates and prodrugs thereof,
where Trp is D-Trp or L-Trp.

In an embodiment of the disclosure, the compound of Formula I has the following structure:

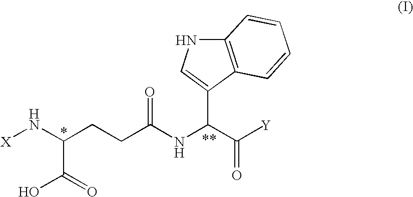

wherein X and Y are as defined above and the stereochemistry at * is L and at ** is D or L.

The present disclosure further includes a pharmaceutical composition comprising one or more compounds of the disclosure and a pharmaceutically acceptable carrier. In particular the pharmaceutical composition is formulated for oral administration.

In another embodiment, the disclosure provides a method of regulating the immune system and/or hemopoiesis comprising administering to the animal in need thereof, an effective amount of one or more compounds of the disclosure. In one embodiment, the immune system in an animal is stimulated using the method of the disclosure. In yet another embodiment, hemopoiesis is restored in an animal using the method of the disclosure.

Also included in the present disclosure is a use of one or more compounds of the disclosure as a medicament. In an embodiment, the present disclosure includes a use of one or more compounds of the disclosure to regulate the immune system and/or hemopoiesis, as well as a use of one or more compounds of the disclosure to prepare a medicament to regulate the immune system and/or hemopoiesis. Also included in the present disclosure is one or more compounds of the disclosure for use as a medicament for example to regulate the immune system and/or hemopoiesis.

In yet another embodiment, the disclosure includes a method of treating hemopoietic disorders, for example, without limitation to immune cytopenia, multiple myeloma, chronic lymphoid leukosis, lymphocytic lymphomas, lymphosarcomas and in particular to β-cellular lymphoid leucosis, comprising administrating to an animal in need thereof, an effective amount of one or more compounds of the disclosure.

The present disclosure also includes a use of one or more compounds of the disclosure to treat hemopoietic disorders. Also included is a use of one or more compounds of the disclosure to prepare a medicament to treat hemopoietic disorders, as well as one or more compounds of the disclosure for use to treat hemopoietic disorders.

Other, features and advantages of the present disclosure will become apparent from the following detailed description. It should be understood, however, that the detailed description and the specific examples, while indicating preferred embodiments of the disclosure are given by way of illustration only, since various changes and modifications within the spirit and scope of the disclosure will become apparent to those skilled in the art from this detailed description.

### DETAILED DESCRIPTION OF THE DISCLOSURE

### I. Definitions

The term “C₁₋₄alkyl” as used herein means straight and/or branched chain, saturated alkyl radicals containing from one to 4 carbon atoms and includes methyl, ethyl, propyl, isopropyl, n-butyl, s-butyl, isobutyl and t-butyl.

The following standard abbreviations for the amino acid residues are used throughout the specification: Glu—glutamic acid; γ-Glu—gamma glutamic acid; Ile—isoleucine; Leu—leucine and Trp—tryptophan. The terms “L” and “D” refer to the stereochemistry of the central carbon of the particular amino acid. Unless otherwise indicated, the stereochemistry of this carbon is L.

The term “solvate” as used herein means a compound of Formula I, or a salt or prodrug of a compound of Formula I, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term “prodrug” as used herein refers to any compound that can be converted in vivo to provide the bioactive agent (i.e., the compound of Formula I). Various forms of prodrugs are well known in the art. A comprehensive description of prodrugs and prodrug derivatives appears in: The Practice of Medicinal Chemistry, Camille G. Wermuth et al., Ch 31, (Academic Press, 1996); Hydrolysis in Drug and Prodrug Metabolism, Bernard Testa and Joachim M. Mayer, (Wiley-VCH, 2003); Design of Prodrugs, edited by H. Bundgaard, (Elsevier, 1985); A Textbook of Drug Design and Development, P. Krogsgaard-Larson and H. Bundgaard, eds. Ch 5, pp. 113-191 (Harwood Academic Publishers, 1991).

The term “compound(s) of the disclosure” as used herein means compound(s) of Formula I, and/or salts, solvates and/or prodrugs thereof.

The term “pharmaceutically acceptable salt” means an acid addition salt or a basic addition salt which is suitable for or compatible with the treatment of animals.

The term “pharmaceutically acceptable acid addition salt” as used herein means any non-toxic organic or inorganic salt of any base compound of the disclosure, or any of its intermediates. Basic compounds of the disclosure that form an acid addition salt include, for example, containing a basic amino group. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts are formed, and such salts exist in either a hydrated, solvated or substantially anhydrous form. In general, the acid addition salts of the compounds of the disclosure are more soluble in water and various hydrophilic organic solvents, and generally demonstrate higher melting points in comparison to their free base forms. The selection of the appropriate salt is known to one skilled in the art. Other non-pharmaceutically acceptable acid addition salts, e.g. oxalates, are used, for example, in the isolation of the compounds of the disclosure, for laboratory use, or for subsequent conversion to a pharmaceutically acceptable acid addition salt.

The term “pharmaceutically acceptable basic addition salt” as used herein means any non-toxic organic or inorganic base addition salt of any acid compound of the disclosure, or any of its intermediates. Acidic compounds of the disclosure that form a basic addition salt include, for example, those comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt is known to a person skilled in the art. Other non-pharmaceutically acceptable basic addition salts, are used, for example, in the isolation of the compounds of the disclosure, for laboratory use, or for subsequent conversion to a pharmaceutically acceptable acid addition salt.

The term a “therapeutically effective amount”, “effective amount” or a “sufficient amount” of a compound of the present disclosure is a quantity sufficient to, when administered to the animal, including a mammal, for example a human, effect beneficial or desired results, including clinical results, and, as such, an “effective amount” or synonym thereto depends upon the context in which it is being applied. For example, in the context of regulating the immune system or hemopoiesis, it is an amount of the compound sufficient to achieve such regulation as compared to the response obtained without administration of the compound. The amount of a given compound of the present disclosure that will correspond to such an amount will vary depending upon various factors, such as the given drug or compound, the pharmaceutical formulation, the route of administration, the type of disease or disorder, the identity of the subject or host being treated, and the like, but nevertheless is routinely determined by one skilled in the art. Also, as used herein, a “therapeutically effective amount” of a compound of the present disclosure is an amount which regulates the immune system or hemopoiesis (e.g., as determined by clinical symptoms) in a subject as compared to a control. As defined herein, a therapeutically effective amount of a compound of the present disclosure is readily determined by one of ordinary skill by routine methods known in the art.

In an embodiment, a therapeutically effective amount of a compound of the present disclosure ranges from about 0.001 to about 0.1 mg/kg body weight. The skilled artisan will appreciate that certain factors influence the dosage required to effectively treat an animal and these factors include, but are not limited to, the severity of the disease or disorder, previous treatments, the general health and/or age of the subject and other diseases present.

Moreover, a “treatment” or “prevention” regime of an animal with a therapeutically effective amount of the compound of the present disclosure consists of a single administration, or alternatively comprise a series of applications. For example, the compound(s) of the present disclosure are administered at least once a week. However, in another embodiment, the compound(s) are administered to the subject from about one time per week to about four times daily for a given treatment. The length of the treatment period depends on a variety of factors, such as the severity of the disease or disorder, the age of the patient, the concentration and the activity of the compound(s) of the present disclosure, or a combination thereof. It will also be appreciated that the effective dosage of the compound used for the treatment or prophylaxis may increase or decrease over the course of a particular treatment or prophylaxis regime. Changes in dosage result and become apparent by standard diagnostic assays known in the art. In some instances, chronic administration is required.

As used herein, and as well understood in the art, “treatment” is an approach for obtaining beneficial or desired results, including clinical results. Beneficial or desired clinical results include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” also means prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with an immune system disorder and/or a hemopoiesis disorder or manifesting a symptom associated therewith.

As used herein, “administered contemporaneously” means that two substances are administered to an animal such that they are both biologically active in the animal at the same time. The exact details of the administration will depend on the pharmacokinetics of the two substances in the presence of each other, and include, for example, administering one substance within 24 hours of administration of the other, if the pharmacokinetics are suitable. Designs of suitable dosing regimens are routine for one skilled in the art. In particular embodiments, two substances are administered substantially simultaneously, i.e. within minutes of each other, or in a single composition that comprises both substances.

To “modulate” a function or activity, such as immune response or hemopoiesis, is to change the function or activity when compared to otherwise same conditions except for a condition or parameter of interest, or alternatively, as compared to another conditions.

Hematopoiesis or hemopoiesis is the formation of cellular components.

The term “animal”, as used herein includes all members of the animal kingdom, especially mammals, including human. The subject or patient is suitably a human.

The term “a cell” as used herein includes a plurality of cells. Administering a compound to a cell includes in vivo, ex vivo and in vitro treatment.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### II. Compounds of the Disclosure

Peptides of the Formula I comprising a L-γ-glutamic acid-tryptophan residue have been prepared and surprisingly shown to have good oral activity where the corresponding peptide comprising the more common L-α-glutamic acid-tryptophan had no activity when administered by the oral route.

Accordingly, the present disclosure includes a compound selected from a compound of the Formula I:

X-L-γ-Glu-Trp-Y (I)

wherein
X is selected from H, C₁₋₄alkyl, C(O)C₁₋₄alkyl, L-Leu, L-Ile and L-Trp;
Y is selected from OH, NH₂, NHC₁₋₄alkyl, N(C₁₋₄alkyl)(C₁₋₄alkyl), L-Leu and L-Ile; and
and pharmaceutically acceptable salts, solvates and prodrugs thereof,
where Trp is D-Trp or L-Trp.

In an embodiment of the disclosure, the compound of Formula I has the following structure:

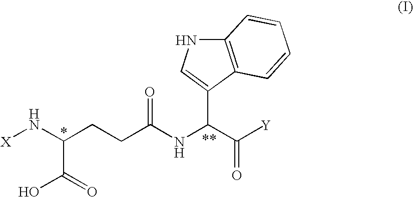

wherein X and Y are as defined above and the stereochemistry at * is L and at ** is D or L.

In an embodiment of the disclosure, X is selected from H, C(O)CH₃, L-Leu and L-Ile. In another embodiment of the disclosure, Y is selected from OH, L-Leu and L-Ile.

In a further embodiment of the disclosure, the compound of Formula I is a tripeptide so that when X is selected from L-Leu, L-Ile or L-Trp, Y is selected from OH, NH₂, NHC₁₋₄alkyl and N(C₁₋₄alkyl)(C₁₋₄alkyl) and when X is selected from H, C₁₋₄alkyl and C(O)C₁₋₄alkyl, Y is selected from L-Leu and L-Ile.

In an embodiment of the disclosure, the compounds of Formula I are selected from

H-L-Ile-L-γ-Glu-L-Trp-OH;

H-L-γ-Glu-D-Trp-L-Ile-OH;

H-L-γ-Glu-L-Trp-L-Ile-OH;

H-L-Leu-L-γ-Glu-L-Trp-OH; and

pharmaceutically acceptable salts, solvates and prodrugs thereof.

In another embodiment, the compound of the present disclosure is H-Ile-γ-Glu-Trp-OH, or a salt, solvate or prodrug thereof.

All of the compounds of Formula I have at least two asymmetric centers therefore, they may exist as diastereomers. It is to be understood that all such isomers and mixtures thereof in any proportion are encompassed within the scope of the present disclosure. Compounds of the present disclosure containing asymmetrically substituted atoms are in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

### III. Methods of Preparing the Compounds of the Disclosure

The compounds of the disclosure are prepared by chemical synthesis using techniques known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

According to an embodiment of the present disclosure, the compound of the Formula (I) is synthesized by step-by-step building of the peptide chain beginning with the C-terminal amino acid. The process involves maximum blocking of functional groups, starting from an amino acid alkyl ester, using the method of active esters and the method of mixing anhydrides, suitably using a t-butyloxycarbonyl group as the amino protecting group.

In a suitable embodiment, the method involves the blocking of the amino, carboxyl and other reactive side groups of the amino acid(s) which are known to react during the synthesis. Suitable blocking agents are known to a person skilled in the art. For example, a suitable carboxy blocking agent include, without limitation, ethyl, nitrobenzyl, and t-butyl. A suitable amino blocking agent include, without limitation, carbobenzoxy, tosyl, trifluoracetyl and, suitably, t-butyloxycarbonyl (Boc). The amino acids are then coupled and the blocking agents subsequently removed. The peptide is optionally further purified using reverse phase chromatography.

In an embodiment of the disclosure, the C-terminal amino acid is blocked at its amino terminal end, suitably with t-butyloxycarbonyl and coupled to another molecule, such as pentafluorophenol, to form an amino acid alkyl-ester. This occurs by chilling the mixture of the protected amino acid and pentaflorophenol in ethylacetate to about −5° C. and adding N,N-dicyclohexylcarbodiimide. The mixture is then stirred at room temperature for three hours, the forming N,N-dicyclohexylurea removed by filtration, the remains crystallized in ethylacetate-hexane and the residue filtered out. The amino protected alkyl-ester amino acid is then coupled to another amino acid or peptide, such as γ-Glu-Trp, in the presence of dimethylformamide, to give an amino protected peptide, such as Boc-Ile-γ-Glu-Trp-OH. The blocking agent, Boc, is then removed using conventional methods known in the art and the peptide purified using reverse phase HPLC chromatography.

The peptides of the disclosure are optionally labelled using conventional methods with various enzymes, fluorescent materials, luminescent materials and radioactive materials. Suitable enzymes, fluorescent materials, luminescent materials, and radioactive material are well known to a person skilled in the art.

Peptides of the disclosure are converted into pharmaceutical salts by reacting with inorganic acids including, without limitation, hydrochloric acid, sulfuric acid, hydrobromic acid, phosphoric acid, or organic acids including formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The methods described above result in the formation of the corresponding free acid and/or free amine or one or both of the corresponding salts thereof. This will depend on the reaction conditions and final isolation procedures as would be known to a person skilled in the art. The formation of, or transformation to, a desired compound salt is achieved using standard techniques. For example, the neutral compound is treated with an acid or base in a suitable solvent and the formed salt is isolated by filtration, extraction or any other suitable method.

The formation of solvates of the compounds of the disclosure will vary depending on the compound and the solvate. In general, solvates are formed by dissolving the compound in the appropriate solvent and isolating the solvate by cooling or using an antisolvent. The solvate is typically dried or azeotroped under ambient conditions.

Prodrugs of the compounds of Formula I are, for example, conventional esters formed with available hydroxy, amino or carboxyl groups. For example, available hydroxy or amino groups are acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

In some cases the chemistries outlined above are modified, for instance by use of protective groups, to prevent side reactions due to reactive groups, such as reactive groups attached as substituents. This is achieved by means of conventional protecting groups, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999.

### III. Uses/Methods

The present disclosure offers peptides that are used for experimental purposes and in medicine. The peptides and pharmaceutical compositions containing the peptides have significant immunoregulatory effects and accordingly are used to promote or suppress recognition and destruction of abnormal or mutant cell types or antigens which arise within the body. The utility of the peptides of the present disclosure is seen in more detail with reference to the Examples.

In one aspect, the disclosure provides a method of modulating the immune system and/or hemopoiesis in an animal in need thereof comprising administering to the animal an effective amount of one or more compounds of the disclosure. In one embodiment, the disclosure provides a method of stimulating the immune system of an animal. In another embodiment, the disclosure provides a method of restoring hemopoiesis in an animal with impaired hemopoiesis, for example caused by irradiation or cytostatic agents.

In yet another embodiment the disclosure provides a method of treating hemopoietic disorders, for example, without limitation to immune cytopenia, multiple myeloma, chronic lymphoid leukosis, lymphocytic lymphomas, lymphosarcomas and in particular b-cellular lymphoid leucosis.

In another embodiment the disclosure provides a method for treating immune and/or hemopoietic disorders such as cancer in an animal comprising administering to the animal an effective amount of one or more compounds of the disclosure, suitably in combination with a cytostatic agent. In an embodiment, the cytostatic agent is either oxyurea or hyperthermia.

The present disclosure includes a use of one or more compounds of the disclosure as a medicament.

Also included in the present disclosure is a use of one or more compounds of the disclosure to regulate the immune system and/or hemopoiesis. as well as a use of one or more compounds of the disclosure to prepare a medicament to regulate the immune system and/or hemopoiesis.

The present disclosure also includes one or more compounds of the disclosure for use as a medicament, for example to regulate the immune system and/or hemopoiesis.

The present disclosure also includes a use of one or more compounds of the disclosure to treat a hemopoietic disorder. Also included is a use of one or more compounds of the disclosure to prepare a medicament to treat a hemopoietic disorder as well as one or more compounds of the disclosure for use to treat a hemopoietic disorder.

In an embodiment the hemopoietic disorder is selected from immune cytopenia, multiple myeloma, chronic lymphoid leukosis, lymphocytic lymphomas, lymphosarcomas and in particular b-cellular lymphoid leucosis.

### Compositions

According to another aspect of the present disclosure, there is included a pharmaceutical composition comprising one or more compounds selected from a compound of Formula I as defined above, and pharmaceutically acceptable salts, solvates, and prodrugs thereof, and a pharmaceutically acceptable carrier.

The compounds of the disclosure are suitably formulated into pharmaceutical compositions for administration to animals in a biologically compatible form suitable for administration in vivo.

The compositions containing the compounds of the disclosure are prepared by known methods for the preparation of pharmaceutically acceptable compositions which are administered to subjects, such that an effective quantity of the active substance is combined in a mixture with a pharmaceutically acceptable vehicle. Suitable vehicles are described, for example, in Remington's Pharmaceutical Sciences (2003—20th edition) and in The United States Pharmacopeia: The National Formulary (USP 24 NF19) published in 1999. On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of Formula I are used pharmaceutically in the form of the free base, in the form of salts, solvates and as hydrates. All forms are within the scope of the disclosure. Acid and basic addition salts are formed with the compounds of the disclosure for use as sources of the free base form even if the particular salt per se is desired only as an intermediate product as, for example, when the salt is formed only for the purposes of purification and identification. All salts that can be formed with the compounds of the disclosure are therefore within the scope of the present disclosure.

In accordance with the methods of the disclosure, the described compounds of the disclosure are administered to a patient in a variety of forms depending on the selected route of administration, as will be understood by those skilled in the art. The compounds of the disclosure are administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration is by continuous infusion over a selected period of time. Suitably the compounds of the disclosure are administered by the oral route. According the composition of the disclosure is suitably formulated for oral delivery.

In an embodiment of the disclosure the pharmaceutical composition contains about 0.01% to about 1%, suitably about 0.01% to about 0.5%, of one or more compounds of the present disclosure. The composition is prepared, for example, by mixing the carrier and the compound(s) at a temperature of about 40° C. to about 70° C., the composition retains stability in solution for 3 years at temperatures up to about 25° C.

In an embodiment, a compound of the disclosure is orally administered, for example, with an inert diluent or with an assimilable edible carrier, or it is enclosed in hard or soft shell gelatin capsules, or it is compressed into tablets, or it is incorporated directly with the food of the diet. For oral therapeutic administration, the compound of the disclosure is incorporated with excipient and used in the form of ingestible tablets, powders, buccal tablets, troches, capsules, elixirs, suspensions, syrups, wafers, and the like. The ratio of compound to excipient is, for example, about 0.01% to about 1%.

In another embodiment, a compound of the disclosure is administered parenterally. Solutions of a compound of the disclosure are prepared in water suitably mixed with a surfactant such as hydroxypropylcellulose. Dispersions are also prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

The pharmaceutical forms suitable for injectable use include sterile aqueous, normal saline, salt or buffered solutions or dispersion and sterile powders for the extemporaneous preparation of sterile injectable solutions or dispersions. In all cases the form must be sterile and must be fluid to the extent that easy syringability exists. The ratio of compound to solvent is, for example, about 0.001% to about 0.1%. Injectable compositions are prepared, for example, by adding the appropriate amount of solvent to an accurately weighed amount of the compound powder. The solution is then filtered, sterilized, bottled or ampouled.

Compositions for nasal administration are conveniently formulated as aerosols, drops, gels or powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is for example, a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compounds of the disclosure, are administered to an animal, suitably a human patient, alone or in combination with pharmaceutically acceptable carriers, as noted above, the proportion of which is determined by the solubility and chemical nature of the compound, chosen route of administration and standard pharmaceutical practice.

The compounds of the disclosure, are formulated alone or for contemporaneous administration with other agents that modulate immune responses or hemopoiesis, or in combination with other types of treatment for immune disorders or hemopoietic disorders. Therefore, according to yet another aspect of the present disclosure, there is included a pharmaceutical composition comprising one or more compounds selected from a compound of Formula I, and pharmaceutically acceptable salts, solvates, and prodrugs thereof, and a pharmaceutically acceptable carrier, for the preparation of a medicament for the treatment of immune disorders or hemopoietic disorders, optionally to be used contemporaneously with another agent to treat immune disorders or hemopoietic disorders.

The following non-limiting examples are illustrative of the present disclosure:

### Examples

### Example 1

### Preparation of H-L-Ile-L-Glu-L-Trp-OH

### (a) Preparation of Boc-L-Glu(OBzl)-L-Trp-OMe

16.9 g (0.05 mol) of Boc-L-Glu(OBzl)-OH was dissolved in dioxane. 18.5 g (0.058 mol) of O-(1H-Benzotriazo-1-yl)-N,N,N′,N′-tetramethyl-uronium tetrafluoroborate (TBTU) was then added to the solution and mixed well. 12.7 g (0.05 mol) of L-Trp-OMe.HCl and 25.3 ml (0.25 mol) of N-methylmorpholine (to pH ˜9-9.2) were also then added to the mixture. The suspension dissolved during the completion of the reaction after 12-18 hours at room temperature.

The solvents were evaporated in vacuo and the residual oil was dissolved in 250 ml of EtOAc, transferred into a separatory funnel and washed with 50 ml of 5% H₂SO₄, 2×50 ml of water, 150 ml of 5% NaHCO₃, and 3×50 ml of water to a neutral pH. The organic layer was separated and dried with anhydrous sodium sulfate. After drying, the EtOAc was evaporated in vacuo.

The residue was dissolved in the mixture of 150 ml of ethyl ether and 60 ml of hexane. A precipitate was formed, filtered off and washed with a mixture of 100 ml of ethyl ether and 50 ml of hexane and subsequently dried.

The yield was 21.5 g (79.9%) and had an R_{f}=0.83 (CHCl₃:EtOAc:MeOH:AcOH=6:3:1:0.1).

### (b) Preparation of Fmoc-L-Ile-L-Glu(OBzl)-L-Trp-OMe

26.9 g (0.05 mol) of Boc-L-Glu(OBzl)-L-Trp-OMe was dissolved in 50 ml of dichloromethane. 50 ml of trifluoroacetic acid was added to the solution and the mixture was stirred for 40 min at room temperature. The solvent was evaporated in vacuo and the residual oil was dissolved in dioxane. N-methylmorpholine was then added to the mixture to a pH ˜9-9.2. (Solution 1.)

16.9 g (0.048 mol) of Fmoc-L-Ile-OH was dissolved in dioxane. 19.9 g (0.062 mol) of O-(1H-Benzotriazo-1-yl)-N,N,N′,N′-tetramethyl-uronium tetrafluoroborate (TBTU) was added to the solution and mixed well. Solution 1 was then added to the mixture. The suspension dissolved during the completion of reaction after 12-18 hours at room temperature.

Solvents were evaporated in vacuo and the residual oil was dissolved in 250 ml of EtOAc, transferred into a separatory funnel and washed with 2×75 ml of 5% H₂SO₄, 3×50 ml of water, 150 ml of 5% NaHCO₃, and 3×50 ml of water to a neutral pH. The organic layer was separated and dried with anhydrous sodium sulfate. After drying, the EtOAc was evaporated in vacuum.

The residue was dissolved in 200 ml of hot EtOAc. A mixture of 300 ml of ethyl ether and 200 ml of hexane was then added to the solution.

A precipitate was formed, filtered off, and washed with a mixture of 50 ml of ethyl ether and 50 ml of hexane and subsequently dried.

The yield was 28.5 g (73.8%) and had an R_{f}=0.85 (CHCl₃:EtOAc:MeOH=6:3:1).

### (c) Preparation of H-L-Ile-L-Glu-L-Trp-ONa

100 ml of dichloromethane and 120 ml of isopropanol were added to 19.4 g (0.025 mol) of -L-Ile L-Glu(OBzl)-L-Trp-OMe. 24 ml of 3N NaOH was then added to the mixture. The suspension dissolved during the completion of the reaction after 3-4 hours at room temperature. The solvents were then evaporated in vacuo and the residual oil was dissolved in 200 ml of EtOAc and 200 ml of water, and transferred into a separatory funnel. The water layer was washed with 100 ml of EtOAc and separated and the pH of the solution was adjusted to 6.2 with acetic acid. The water solution was then evaporated in vacuo to a minimum volume. 600 ml of ethanol was then added to the residue. A precipitate was formed, filtered off, washed with ethanol and then dried.

The yield was 8.9 g (76.0%) and had an R_{f}=0.53 (CHCl₃:MeOH:32% AcOH=5:3:1).

In a like manner, the following additional compounds were prepared:

H-L-Ile-L-Glu-L-Trp-OH

H-L-Ile-γ-Glu-L-Trp-OH

H-Ile-D-Glu-(D-Trp)-OH

H-L-γ-Glu-D-Trp-L-Ile-OH

H-L-γ-Glu-L-Trp-L-Ile-OH

H-L-Leu-L-Glu-L-Trp-OH and

H-L-Leu-L-γ-Glu-L-Trp-OH

### Example 2

This example sets out physical organic data with respect to various embodiments of the compounds of the present disclosure. Table 1 contains Rf₁ (in chloroform-methanol-32% acetic acid=60:45:20) and Rf₂ (in butanol-pyridine-water-acetic acid=5:5:4:1) values for a number of compounds Formula I.

### Example 3

The biological activity of the novel peptides was compared to that of H-L-Ile-L-Glu-L-Trp-OH and other reference peptides. The results are presented in Tables 2-4.

### Irradiation

Test animals were irradiated with ⁶⁰Co gamma-rays at a dose rate of 0.8 Gy per min. Bone marrow recipients were irradiated with a dose of 8 Gy; donors were irradiated with a dose of 4 Gy. The suspension of bone marrow cells of intact animals was irradiated with 1 Gy, 5-10 min prior to injection to lethally irradiated recipients.

### A Study of the Peptide Radiotherapeutic Properties and the Effect on Hematopoietic Progenitor Population

Stimulation of CFU-S regeneration in the bone marrow of 1-Gy irradiated mice was studied with different routes of administration of the peptides.

For this purpose, at least 5 mice from each group were killed and their bone marrow was obtained. Cell suspension was prepared and injected to lethally irradiated mice. After 9 days, spleen colonies were counted to determine the CFU-S content in 1-Gy irradiated donors who received the peptide. The results are summarized in Tables 2-4. Table 2 shows results using intraperitoneal administration, while Table 3 shows the results using oral administration. Table 4 shows the effects of different doses and routes of administration of H-L-Ile-L-Glu-L-Trp-OH (reference peptide) and H-L-Ile-L-γ-Glu-L-Trp-OH (peptide of the present disclosure).

The result clearly show that peptides comprising a L-γ-Glu-Trp show oral activity while those that comprise the more common L-Glu-Trp showed now activity when administered orally.

TABLE 1

| Peptide | R_{f1} | R_{f2} |
|---|---|---|
| H-L-Ile-L-Glu-L-Trp-OH | 0.38 | 0.54 |
| (comparison peptide) | | |
| H-L-Ile-γ-Glu-L-Trp-OH | 0.37 | 0.51 |
| H-Ile-D-Glu-(D-Trp)-OH | 0.36 | 0.54 |
| (comparison peptide) | | |
| H-L-γ-Glu-D-Trp-L-Ile-OH | 0.33 | 0.51 |
| H-L-γ-Glu-L-Trp-L-Ile-OH | 0.33 | 0.52 |
| H-L-Leu-L-Glu-L-Trp-OH | 0.33 | 0.52 |
| (comparison peptide) | | |
| H-L-Leu-L-γ-Glu-L-Trp-OH | 0.32 | 0.51 |

TABLE 2

| | | Colony | Stimulation | Activity |
|---|---|---|---|---|
| 1 Gy | Peptide | count | index | % |
| − | Control | 10.2 ± 0.3 | 1 | 100 |
| + | — | 4.6 ± 0.2* | | 0 |
| + | H-L-Ile-L-Glu-L-Trp-OH | 8.2 ± 0.5** | 0.8 | 80 |
| | (comparison peptide) | | | |
| + | H-L-Ile-γ-Glu-L-Trp-OH | 8.6 ± 0.7** | 0.8 | 84 |
| + | H-L-γ-Glu-D-Trp-L-Ile- | 6.2 ± 0.5** | 0.6 | 61 |
| | OH | | | |
| + | H-L-Trp-L-Glu-L-Ile-OH | 8.1 ± 1.0** | 0.8 | 79 |
| + | H-L-γ-Glu-L-Trp-L-Ile- | 7.5 ± 0.5** | 0.7 | 73 |
| | OH | | | |
| + | H-L-Leu-L-Glu-L-Trp- | 7.6 ± 0.7** | 0.7 | 74 |
| | OH | | | |
| + | H-L-Leu-L-γ-Glu-L-Trp- | 6.9 ± 0.6** | 0.7 | 69 |
| | OH | | | |

Note:
The data in the Table are the average of two tests; peptides were injected intraperitoneally in a dose of 100 ug/kg, 30 min after injection of bone marrow suspension.
*P < 0.05 as compared with the intact control;
**P < 0.05 as compared with irradiated animals.

TABLE 3

| | Peptide | Colony | Stimulation | Activity |
|---|---|---|---|---|
| 1 Gy | (per os) | count | index | % |
| − | Control | 10.3 ± 0.6 | 1 | 100 |
| + | — | 5.9 ± 0.3* | | 0 |
| + | H-L-Ile-L-Glu-L-Trp-OH | 5.6 ± 0.5 | 0 | 0 |
| | (comparison peptide) | | | |
| + | H-L-Ile-L-γ-Glu-L-Trp- | 8.6 ± 0.7** | 0.8 | 83 |
| | OH | | | |
| + | H-L-γ-Glu-D-Trp-L-Ile- | 6.8 ± 0.5** | 0.7 | 66 |
| | OH | | | |
| + | H-L-Ile-D-Glu-D-Trp- | 5.8 ± 0.3 | 0 | 0 |
| | OH | | | |
| + | H-L-Trp-L-Glu-L-Ile-OH | 5.9 ± 0.8 | 0 | 0 |
| + | H-L-Glu-L-γ-Trp-Ile-OH | 7.7 ± 0.6** | 0.7 | 75 |
| + | H-L-Leu-L-Glu-L-Trp- | 6.2 ± 0.3 | 0 | 0 |
| | OH | | | |
| + | H-L-Leu-L-γ-Glu-L-Trp- | 6.7 ± 0.7** | 0.7 | 67 |
| | OH | | | |

Note:
The data in the Table are the average of two tests; peptides were given by oral gavages in a dose of 100 ug/kg, 30 min after injection of bone marrow suspension.
*P < 0.05 as compared with the intact control;
**P < 0.05 as compared with irradiated animals.

TABLE 4

| | | Peptide | | | |
|---|---|---|---|---|---|
| Radiation | | dose, | Route of | Colony | % |
| Dose | Peptide | ug/kg | administration | count | stimulation |
| — | — | — | — | 11.9 ± 0.4 | 100 |
| 1 Gy | — | — | — | 6.2 ± 0.6** | 0 |
| 1 Gy | H-L-lle-L-Glu-L-Trp-OH | 10 | IP | 9.7 ± 0.4* | 82 |
| 1 Gy | H-L-lle-L-Glu-L-Trp-OH | 100 | IP | 9.6 ± 0.7* | 81 |
| 1 Gy | H-L-lle-L-Glu-L-Trp-OH | 1000 | IP | 11.8 ± 00.9* | 100 |
| 1 Gy | H-L-lle-L-Glu-L-Trp-OH | 100 | per/os | 6.7 ± 0.5 | 0 |
| 1 Gy | H-L-lle-L-Glu-L-Trp-OH | 1000 | per/os | 6.1 ± 00.3 | 0 |
| 1 Gy | H-L-lle-L-γ-Glu-L-Trp-OH | 10 | IP | 7.6 ± 0.7* | 64 |
| 1 Gy | H-L-lle-L-γ-Glu-L-Trp-OH | 100 | IP | 8.2 ± 00.4* | 69 |
| 1 Gy | H-L-lle-L-γ-Glu-L-Trp-OH | 1000 | IP | 10.4 ± 00.6* | 87 |
| 1 Gy | H-L-lle-L-γ-Glu-L-Trp-OH | 10 | per/os | 6.9 ± 0.7 | 0 |
| 1 Gy | H-L-lle-L-γ-Glu-L-Trp-OH | 100 | per/os | 8.3 ± 0.4* | 70 |
| 1 Gy | H-L-lle-L-γ-Glu-L-Trp-OH | 1000 | per/os | 10.7 ± 00.8* | 90 |

Note:
The data in the Table are the average of two tests; peptides were given
30 min after injection of bone marrow suspension.
*P<0.05 as compared with the intact control;
**P<0.05 as compared with irradiated animals.

1.-23. (canceled)

24. A compound having the formula:
X-L-γ-Glu-Trp-Y
wherein X is selected from H, C₁₋₄alkyl, C(O)C₁₋₄alkyl, L-Leu, L-Ile and L-Trp;
Y is selected from OH, NH₂, NHC₁₋₄alkyl, N(C₁₋₄alkyl)(C₁₋₄alkyl), L-Leu and L-Ile;
Trp is D-Trp or L-Trp, and Glu is L-Glu;
with the proviso that when X is H, C₁₋₄alkyl, or C(O)C₁₋₄alkyl, Y is not —OH.

25. The compound of claim 24, which is a tripeptide.

26. The compound of claim 24, wherein X is selected from L-Leu, L-Ile, and L-Trp.

27. The compound of claim 24, wherein Y is selected from OH, L-Leu and L-Ile.

28. The compound of claim 24, wherein when X is selected from L-Leu, L-Ile or L-Trp, Y is selected from OH, NH₂, NH₁₋₄alkyl and N(C₁₋₄alkyl)(C₁₋₄alkyl).

29. The compound of claim 24, wherein when X is selected from H and C(O)C₁₋₄alkyl, Y is selected from L-Leu and L-Ile.

30. The compound of claim 24, selected from
H-L-Ile-L-γ-Glu-L-Trp-OH;
H-L-γ-Glu-D-Trp-L-Ile-OH;
H-L-γ-Glu-L-Trp-L-Ile-OH; and
H-L-Leu-L-γ-Glu-L-Trp-OH.

31. The compound of claim 24, which is H-Ile-γ-Glu-Trp-OH.

32. A pharmaceutical composition comprising an effective amount of a compound according to claim 24, and a pharmaceutically acceptable carrier.

33. The pharmaceutical composition according to claim 32, formulated for oral administration.

34. The oral formulation of claim 33, which is a tablet, lozenge, or pastille.

35. A method for stimulating hematopoiesis, comprising administering to a person in need thereof a composition according to claim 24.

36. A method for treating immune cytopenia, multiple myeloma, chronic lymphoid leukosis, lymphocytic lymphoma, lymphosarcoma or p-cellular lymphoid leucosis, comprising administering to a person in need thereof a composition according to claim 24.

37. A method for treating cancer, comprising administering to a person in need thereof a composition according to claim 24 in combination with a cytostatic agent.
